(19) United States
(12) Patent Application Publication
(10) Pub. No.: US 2008/0139438 A1
(43) Pub. Date: Jun. 12, 2008

(54) Long-Chain Fatty Alcohol Alkoxylates in Cleaning Preparations

(76) Inventors: Corinna Boehme, Dormagen (DE); Sabine Both, Neuss (DE); Rainer Eskuchen, Langenfeld (DE); Susan Fleet-Brandt, Aachen (DE)

Correspondence Address: COGNIS CORPORATION;PATENT DEPARTMENT, 300 BROOKSIDE AVENUE, AMBLER, PA, 19002, US

(21) Appl. No.: 11/846,098

(22) Filed: Aug. 28, 2007

(30) Foreign Application Priority Data
Sep. 5, 2006 (EP) 06018611.1

(51) Int. Cl. C11D 3/20; C07C 43/03; C11D 3/22; C11D 3/37

(52) U.S. Cl. 510/221; 568/613; 510/475; 510/506; 510/470; 510/220

(57) ABSTRACT

A cleaning preparation for cleaning hard surfaces including at least one alkoxylated fatty alcohol corresponding to general formula (I):

R—O—(CₙH₂ₙO)ₘ—H (I)

in which R is a linear or branched, saturated or unsaturated, alkyl or alkenyl group containing at least 19 carbon atoms, m represents a value of 20 or more, and n represents 2 or 3 is provided. A surfactant mixture including at least one compound corresponding to the above general formula (I), and at least one additional compound is also provided. A liquid rinse agent including water; a solubilizer; and at least one alkoxylated fatty alcohol corresponding to the above general formula (I), where the at least one alkoxylated fatty alcohol corresponding to general formula (I) is present in quantities of from about 0.1 to about 25% by weight of the rinse agent is also provided.

### CROSS-REFERENCE TO RELATED APPLICATIONS

This application claims priority under 35 U.S.C. § 119 from European Patent Application No. 06018611.1, filed Sep. 5, 2006, the entire disclosure of which is hereby incorporated by reference herein.

### BACKGROUND OF THE INVENTION

1. Field of the Invention

This invention relates generally to selected fatty alcohol alkoxylates in cleaning preparations and to cleaning preparations containing the fatty alcohol alkoxylates, preferably cleaning preparations for use in automatic dishwashing processes.

2. Background Information

The cleaning of hard surfaces and particularly the washing of dishes impose particular demands on the preparations used. This applies in particular to automatic dishwashing. The three components of the automatic system are detergent, rinse agent and regenerating salt for softening water. The key functions of the principal constituent, the detergent, are soil separation, soil dispersion, the binding of residual water hardness and corrosion inhibition. Following the trend towards simplified use, many manufacturers today offer their customers multifunctional dish detergents, i.e, the detergent additionally contains rinse agents and water softeners or agents for retaining shine on metal surfaces or for protection against silver discoloration after washing, so that the customer does not have to use separate agents to perform these functions, but instead achieves the desired result with only a single supply form.

Compared with the standard “clear rinse” system (detergent, salt and rinse agent as separate products), conventional multifunctional automatic dish detergents (ADDs) show poorer drying behavior. By drying behavior is meant the extent to which tableware cleaned with a dish detergent still has water, mostly in the form of droplets, on its surface after undergoing the dishwashing process.

The increased use of multifunctional compositions has resulted in a deterioration in drying behavior by comparison with the traditional rinse agent. Accordingly, a search has been conducted to find ways of improving the drying performance of hard surface cleaners and particularly dish detergents. However, the cleaning performance and particularly the clear-rinse performance of the detergents should not be adversely affected by additives. For example, the addition or presence of rinse agents should not result in excessive foaming of the detergent. Ideally, an additive should actually improve the overall performance of the detergent.

A key parameter in dishwashing is clear-rinse performance. This determines the extent of deposits on the items of tableware after washing. The deposits are essentially mineral compounds, more particularly Ca and/or Mg salts, but also surfactant residues. However, it is principally lime which leads to the deposits so disliked by the consumer. In order to reduce the extent of these deposits, conventional dish detergents, particularly automatic dish detergents, generally contain so-called rinse agents. Branded rinse agents are usually mixtures of low-foaming nonionic surfactants, typically fatty alcohol polyethylene/polypropylene glycol ethers, solubilizers (for example cumemesulfonate), organic acids (for example citric acid) and solvents (for example ethanol). The function of the rinse agents is to influence the interfacial tension of the water in such a way that it is able to drain from the tableware in the form of a very thin, coherent film, so that no droplets of water, streaks or films are left behind after the subsequent drying phase. There are two kinds of deposits, namely: spotting, which is caused by drying water droplets, and filming, i.e., layers formed by the drying of thin films of water. Accordingly, it is understandable why there is a continuing demand for improved rinse agents which are expected not only to provide an improvement in clear rinse performance, but also to avoid the practical problems mentioned above.

### SUMMARY OF THE INVENTION

Briefly described, according to an aspect of the invention, a cleaning preparation for cleaning hard surfaces includes at least one alkoxylated fatty alcohol corresponding to general formula (I):

R—O—(CₙH₂ₙO)ₘ—H (I)

in which R is a linear or branched, saturated or unsaturated, alkyl or alkenyl group containing at least 19 carbon atoms, m represents a value of 20 or more, and n represents 2 or 3.

According to another aspect of the invention, a surfactant mixture includes (a) at least one compound corresponding to general formula (I):

R—O—(CₙH₂ₙO)ₘ—H (I)

in which R is a linear or branched, saturated or unsaturated, alkyl or alkenyl group containing at least 19 carbon atoms, m represents a value of 20 or more and n represents 2 or 3, and, (b) at least one compound selected from
(i) compounds corresponding to formula (IIa):

R¹O[CH₂CH₂O]ₓCH₂CH(OM)R² (IIa)
in which R¹ is a linear or branched, alkyl or alkenyl group containing 4 to 22 carbon atoms, or an R²—CH(OH)CH₂ group, in which R² is a linear or branched, alkyl or alkenyl group containing 8 to 16 carbon atoms, x represents a number from 40 to 80, and M is a hydrogen atom or a saturated alkyl group containing 1 to 18 carbon atoms;
(ii) compounds corresponding to formula (IIb):

R³O[CH₂CHCH₃O]_{y}[CH₂CH₂O]_{z}CH₂CH(OH)R⁴ (IIb)
in which R³ is a linear or branched, alkyl or alkenyl group containing 8 to 22 carbon atoms, R⁴ is a linear or branched, alkyl or alkenyl group containing 8 to 16 carbon atoms, y represents a number from 10 to 35, z is 0 or represents a value from 1 to 5, with the proviso that where R³═R¹, and at the same time R⁴═R², z must be at least 1;
(iii) ethoxylated fatty alcohols corresponding to general formula (III):

R⁵—(OC₂H₄)_{z}—OH (III)
in which R⁵ represents linear or branched, alkyl or alkenyl groups containing 8 to 22 carbon atoms, and z represents a number from 1 to 20;
(iv) compounds corresponding to general formula R⁶CO—(OC₂H₄)ₘ—OR⁷, in which R⁶ is an alkyl or alkenyl group containing 7 to 21 carbon atoms, m represents a number from 11 to 100 and R⁷ is a hydrogen atom or a CO—R⁶ group;
(v) alkyl (oligo)glycosides corresponding to the general formula R⁸O-[G]ₚ, in which R⁸ is an alkyl or alkenyl group containing 4 to 22 carbon atoms, G is a sugar unit containing 5 or 6 carbon atoms and p represents a number from 1 to 10;
(vi) betaines;
(vii) compounds corresponding to general formula (IV):

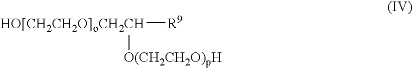

in which R⁹ is a linear or branched, alkyl or alkenyl group containing 4 to 22 carbon atoms, o represents a number from 1 to 20 and p is 0 or represents a number from 1 to 20;
(viii) compounds corresponding to general formula (V):

R¹⁰CH(OR¹¹)CH₂—OR¹¹ (V)
in which R¹⁰ is a saturated or unsaturated, branched or unbranched, alkyl or alkenyl group containing 8 to 16 carbon atoms and the substituents R¹¹ independently of one another represent a group (CH₂CH₂O)ᵣCH₂CH(OH)R², in which r in each of the R¹¹ substituents independently represents 0 or a number from 1 to 50 and R¹⁻² is a saturated or unsaturated, branched or unbranched, alkyl or alkenyl group containing 8 to 16 carbon atoms; and
(ix) compounds corresponding to general formula (VI):

NR¹³₃ (VI)
in which the substituents R¹³ independently of one another represent a group (CH₂CH₂O)ₛ—CH₂CH(OH)R¹⁴, or an alkyl group containing 8 to 16 carbon atoms and s in each substituent R¹³ independently represents 0 or a number from 1 to 50.

According to yet another aspect of the invention, a liquid rinse agent includes water; a solubilizer; and at least one alkoxylated fatty alcohol corresponding to general formula (I):

R—O—(CₙH₂ₙO)ₘ—H (I)

in which R is a linear or branched, saturated or unsaturated, alkyl or alkenyl group containing at least 19 carbon atoms, m represents a value of 20 or more, and n represents 2 or 3, wherein the at least one alkoxylated fatty alcohol corresponding to general formula (I) is present in quantities of from about 0.1 to about 25% by weight.

### DETAILED DESCRIPTION OF THE INVENTION

It has now been found that selected long-chain fatty alcohol alkoxylates are capable of solving the stated problem.

In a first embodiment, the present invention relates to the use of alkoxylated fatty alcohols corresponding to general formula (I):

R—O—(CₙH₂ₙO)ₘ—H (I)

in which R is a linear, branched, saturated or unsaturated alkyl or alkenyl group containing at least 19 carbon atoms, m has a value of 20 or more and n stands for 2 or 3, in hard surface cleaners, more particularly in automatic dish detergents. This includes the use of the alkoxylates corresponding to formula (I) as additives for improving clear rinse performance in hard surface cleaners, more particularly in automatic dish detergents.

“Fatty alcohols” is the generic name for the linear, saturated or unsaturated primary alcohols (1-alkanols) containing 6 to 22 carbon atoms which are obtainable by reduction of the triglycerides, fatty acids or fatty acid methyl esters. Alternatively to their production from fats and oils, fatty alcohols can be obtained by oxo reduction (hydroformylation). In the Alfol process, higher olefins are first produced by oligomerization of ethylene as an organoaluminium compound which, in a further step, is oxidized with atmospheric oxygen and hydrolyzed to the corresponding fatty alcohols.

The alkoxylates of the fatty alcohols are a group of nonionic surfactants which are obtained by alkoxylation, i.e., reaction with ethylene oxide (fatty alcohol ethoxylates, FAEO), propylene oxide or butylene oxide, but preferably by ethoxylation of primary long-chain fatty or oxo alcohols in the presence of basic or acidic catalysts at temperatures of 120 to 200° C. and pressures of 1 to 10 bar. In the reaction of the alcohol with the alkylene oxide, a polyglycol ether mixture of homologous compounds with different degrees of alkoxylation is formed through a polymerization reaction with the starter, fatty alcohol, the distribution of these compounds being variable between a Gauss curve corresponding to statistics and a non-selective Schulz-Flory curve. In the presence of sodium hydroxide for example, a broad homolog distribution is obtained whereas, with alkaline earth metal salts, a “narrow range” homolog distribution is obtained. On account of the formation of 1,4-dioxane as an unwanted secondary product promoted by acidic ethoxylation catalysts and the danger of corrosion affecting the steel of the reactor, basic catalysts, for example sodium methylate in methanol or potassium hydroxide in water, are preferably used on an industrial scale.

The fatty alcohol alkoxylates used in accordance with the invention are long-chained, i.e., the alkyl group or—in the case of unsaturated groups—the alkenyl group has at least 19 carbon atoms, preferred alkoxylates being those of which the alkyl or alkenyl group contains 19 to 30, preferably 20 to 25 and more particularly 21 to 23 carbon atoms. Fatty alcohol alkoxylates based on behenyl alcohol, i.e., 22 carbon atoms in the alkyl chain, are most particularly preferred.

The substituent R in general formula (I) may be linear or branched, although linear alkyl or alkenyl chains are preferred. Accordingly, the substituent R may be both saturated and mono- or polyunsaturated. Compounds corresponding to formula (I) in which the substituent R is saturated are preferably used. In selecting suitable compounds of formula (I), those in which the substituent R is linear and saturated are particularly preferred.

The compounds of formula (I) are alkoxylates. This includes compounds which have been produced by reaction either with ethylene oxide (EO) or propylene oxide, although mixed alkoxylates of EO and PO are also suitable. Compounds of formula (I) in which n=2, i.e., the pure ethoxylates, are particularly preferred. Narrow range alkoxylates can be particularly preferred.

The content of alkoxylates as expressed through the index m in general formula (I) must be at least 20 or greater. Preferred compounds of formula (I) are those in which the index m has a value of 20 to 30, preferably 21 to 28 and more particularly 22 to 26. Alkoxylates containing 25 parts alkoxide, preferably ethylene oxide, per molecule fatty alcohol are most particularly preferred. Other preferred compounds of general formula (I) are those in which R is a linear, saturated alkyl group containing 22 carbon atoms, n has a value of 2 and m may have a value of 22 to 26. A behenyl alcohol which has been reacted with 25 parts ethylene oxide is most particularly preferred.

In a preferred embodiment of the present invention, the above-described alkoxylates of formula (I) are used as sole alkoxylates, i.e., in a detergent for example, alkoxylates of formula (I) are the only alkoxylates present, alkoxylates of formula (I) containing 22 carbon atoms preferably being used.

The alkoxylates used in accordance with the invention are produced in known manner. To this end, the starter alcohol, for example, is introduced into and melted in the reactor. An alkaline catalyst, for example 30% NaOMe, is then introduced into the melt with stirring. The pressure is then reduced, for example to <40 mbar, and the reactor is evacuated at elevated temperature, for example 100 to 120° C., and filled with nitrogen. The requisite quantity of ethylene oxide is then added at further increased temperatures, for example 160° C. to 180° C., and a pressure of at most 5 bar. After the end of the reaction, the reaction mixture is cooled to ca. 100 to 120° C. and the reactor is again evacuated to <40 mbar. The product can be neutralized by addition of stoichiometric quantities of acid (for example lactic acid, acetic acid, citric acid, phosphoric acid) and optionally filtered before bottling.

The alkoxylates of formula (I) are used, for example, as additives in cleaning preparations, preferably in automatic dish detergents. They are used in quantities of 0.1 to 25% by weight, preferably in quantities of 0.5 to 10% by weight and more particularly in quantities of 1.0 to 5% by weight, based on the total weight of the preparation. The alkoxylates may advantageously be used in solid cleaning preparations. However, it is immaterial whether the preparations are formulated as powders, granules, pellets, flakes or shaped bodies, i.e., tablets, or for example as blocks.

However, the fatty alcohol alkoxylates of general formula (I) are not only suitable for use in cleaning preparations or dish detergents. They may also be used, and are preferably used, in rinse agents which may be both solid and liquid rinse agents.

The alkoxylates may also be used with advantage in the form of compounds, preferably in combination with nonionic surfactants. Nonionic surfactants from the group of polyethylene glycols, alkyl (oligo)glycosides, fatty alcohol alkoxylates except the compounds of formula (I), polyol hydroxyalkyl ethers and/or hydroxy mixed ethers are preferred. To this end, the alkoxylates of formula (I) are suitably compounded with the other compounds and may then be further processed in this form. Now, the present invention additionally makes use of the observation that the presence of the selected fatty alcohol alkoxylates of general formula (I) as component a) in combination with structurally different surface-active compounds of type b) can have advantageous properties in regard to the drying behavior and/or clear rinse performance of hard surface cleaners, more particularly dish detergents. At least two surface-active compounds are combined, of which one component a) must be a fatty alcohol alkoxylate of the type described above and the other component b) is selected from one or more of compounds (b)(i) to (b)(ix) described in the following.

Surface-Active Compounds of Type (b)(i)

These compounds are so-called hydroxy mixed ethers (HMEs). Hydroxy mixed ethers correspond to the broad general formula R′O[AO]ₓCH₂CH(OM)R″, in which R′ is a linear or branched alkyl and/or alkenyl group containing 4 to 22 carbon atoms, R″ is a linear or branched alkyl and/or alkenyl group containing 2 to 22 carbon atoms, x has a value of 10 to 80, AO is an ethylene oxide, propylene oxide or butylene oxide group and M can be a hydrogen atom or an alkyl or alkenyl group. Hydroxy mixed ethers of the type in question are known from the literature and are described, for example, in German patent application DE 19738866. They are prepared, for example, by reaction of 1,2-epoxyalkanes (R²CHOCH₂), where R is an alkyl and/or alkenyl group containing 2 to 22 and more particularly 6 to 16 carbon atoms, with alkoxylated alcohols. Hydroxy mixed ethers preferred for the purposes of the invention are those derived from alkoxylates of monohydric C₄₋₁₈ alcohols with the formula R′—OH, R being an aliphatic, saturated, linear or branched alkyl group, more particularly containing 6 to 16 carbon atoms. Examples of suitable straight-chain alcohols are butan-1-ol, caproic alcohol, oenanthic alcohol, caprylic alcohol, pelargonic alcohol, capric alcohol, undecan-1-ol, lauryl alcohol, tridecan-1-ol, myristyl alcohol, pentadecan-1-ol, palmityl alcohol, heptadecan-1-ol, stearyl alcohol, nonadecan-1-ol, arachidyl alcohol, heneicosan-1-ol, behenyl alcohol and the technical mixtures thereof obtained in the high-pressure hydrogenation of technical methyl esters based on fats and oils. Examples of branched alcohols are so-called oxo alcohols which generally contain 2 to 4 methyl groups as branches and are produced by the oxo process and so-called Guerbet alcohols which are branched in the 2-position by an alkyl group. Suitable Guerbet alcohols are 2-ethyl hexanol, 2-butyl octanol, 2-hexyl decanol and/or 2-octyl dodecanol. The alcohols are used in the form of their alkoxylates which are prepared in known manner by reaction of the alcohols with ethylene oxide.

There are also other known hydroxy mixed ethers, namely those which contain more than one free hydroxyl group in the molecule. Such compounds can be prepared, for example, by reacting diols, preferably alkylene glycols and derivatives thereof, preferably polyethylene glycols, with two mols of an alkyl epoxide (R—CHOCH₂) per mol of the diol.

Surface-Active Compounds of Type (b)(i) Corresponding to Formula (IIa):

The compounds are commercially available surfactants of the HME type corresponding to general formula (IIa):

R¹O[CH₂CH₂O]ₓCH₂CH(OM)R² (IIa)

in which R¹ is a linear or branched alkyl and/or alkenyl group containing 4 to 22 carbon atoms or an R²—CH(OH)CH₂ group, where R² is a linear or branched alkyl and/or alkenyl group containing 8 to 16 carbon atoms, x is a number of 40 to 80 and M is a hydrogen atom or a saturated alkyl group containing 1 to 18 carbon atoms. Compounds of type (b)(i) corresponding to general formula (IIa) which contain at least one free hydroxyl group (=—OH) are advantageously used.

Hydroxy mixed ethers derived from ethoxylates of monohydric alcohols with the formula R¹—OH(R¹=a linear alkyl group, x=40 to 60) containing 6 to 18 carbon atoms, preferably 6 to 16 and more particularly 8 to 10 carbon atoms are preferred for the purposes of the invention. Other compounds of general formula (IIa) preferably present in the mixtures according to the invention are those in which the index x is a number of 40 to 70, preferably 40 to 60 and more particularly 40 to 50 and M is a hydrogen atom. Hydroxy mixed ethers of type (b)(i) corresponding to formula (IIa), in which R¹ is an alkyl group containing 8 to 10 carbon atoms, more particularly based on a native fatty alcohol, R² is an alkyl group containing 10 carbon atoms, more particularly a linear alkyl group, and x has a value of 40 to 60, are most particularly preferred. Other preferred mixtures are those which contain a compound of general formula (IIa), in which R¹ is an alkyl and/or alkenyl group containing 8 to 10 carbon atoms and R² is an alkyl or alkenyl group containing 8 to 10 carbon atoms and x has a value of 40 to 50, M again being a hydrogen atom, as the surface active compound of type (b)(i). However, compounds corresponding to general formula (IIa), in which R¹ is an alkyl or alkenyl group containing 8 to 10 carbon atoms, R² is an alkyl group containing 8 to 12 carbon atoms and M is a saturated alkyl group containing 1 to 6 and preferably 1 to 4 carbon atoms, are also suitable as the compounds of type (b)(i). Compounds of the latter type do not contain any free hydroxyl groups, the hydroxyl functions having been alkylated with suitable reagents, for example alkyl halides.

Surface-Active Compounds of Type (b)(ii) Corresponding to Formula (IIb)

Also suitable and preferred are group (b)(ii) compounds corresponding to formula (IIb):

R³O[CH₂CHCH₃O]_{z}[CH₂CH₂O]_{y}CH₂CH(OH)R⁴ (IIb)

in which R³ is a linear or branched alkyl and/or alkenyl group containing 8 to 22 carbon atoms, R⁴ is a linear or branched alkyl and/or alkenyl group containing 8 to 16 carbon atoms, y is a number of 10 to 35, z is 0 or must have a value of 1 to 5. It can be of advantage if, where R³═R¹ and at the same time R⁴═R², the compounds of formula (b)(i) and (ii) selected are those in which the index x is at least 1. If mixtures of the surface-active compounds of type a) with those of type (b)(i) and/or (ii) are used, only those mixtures in which the molecules are structurally different from one another correspond to the technical teaching of the present invention. In other words, structurally different compounds must always be present alongside one another. Particularly preferred compounds of type (b)(ii) are, for example, those in which, in formula (IIb), the index y is a number of 20 to 30 and preferably 20 to 25. Other preferred compounds of type (b)(ii) are those in which, in formula (IIb), R³ is an alkyl group containing 8 to 12 and preferably 8 to 10 carbon atoms, R⁴ is an alkyl group containing 10 to 12 and preferably 10 carbon atoms, y is a number of 15 to 35, preferably 20 to 30, and z is a number of 1 to 3, preferably the number 1.

Other preferred mixtures are those which contain a compound of general formula (IIb), in which R³ is an alkyl and/or alkenyl group containing 11 to 18 carbon atoms and R⁴ is an alkyl or alkenyl group containing 8 to 10 carbon atoms and y is a number of 20 to 35, as the surface-active compound of type (b)(ii).

Mixtures containing a compound of general formula (IIb), in which R³ is an alkyl and/or alkenyl group containing 8 to 12 carbon atoms and R⁴ is an alkyl or alkenyl group containing 8 to 10 carbon atoms, y is a number of 20 to 35 and z is a number of 1 to 3, as the surface-active compound of type (b)(ii) also represent preferred mixtures. The compounds of type (b)(ii) are also hydroxy mixed ether derivatives which can be prepared by ring-opening reaction of propoxylated and/or ethoxylated fatty alcohols with alkyl epoxides in alkaline medium. With derivatives of type (b)(ii) and with all other mixed alkoxylates mentioned herein, i.e., alkoxylates which contain both a propylene oxide unit CH₂CHCH₃O(PO) and an ethylene oxide unit CH₂CH₂O(EO), it is possible that, in the direction of the C atom with the free hydroxyl group, first the EO groups and then the PO groups are arranged blockwise, the opposite sequence (first PO, then EO) also being possible. In addition, the alkoxide groups may also be present in statistical distribution (randomized) in the molecule. Both block alkoxylates and random alkoxylates may also be used alongside one another.

Surface-Active Compounds of Type (b)(iii)

These compounds are fatty alcohol ethoxylates known per se corresponding to general formula (III) R⁵—(OC₂H₄)ₙ—OH, in which R⁵ represents linear or branched alkyl and/or alkenyl groups containing 8 to 22 carbon atoms and z is a number of 1 to 20, preferably 1 to 15 and more particularly 1 to 10. Typical examples are the adducts of on average 1 to 20 mol caproic alcohol, caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol and the technical mixtures thereof obtained, for example, in the high-pressure hydrogenation of technical methyl esters based on fats and oils or aldehydes from Roelen's oxo synthesis and as monomer fraction in the dimerization of unsaturated fatty alcohols. Adducts of 10 to 40 mol ethylene oxide with technical C₁₂₋₁₈ fatty alcohols, such as for example coconut oil, palm oil, palm kernel oil or preferably tallow fatty alcohol, are preferred. Particularly preferred fatty alcohol ethoxylates are based on tallow fatty alcohols ethoxylated with 2 to 10 and preferably 2 to 5 mol ethylene oxide per mol alcohol.

Surface-Active Compounds of Type (b)(iv)

These compounds are mono- and/or preferably diesters of glycol and especially polyglycols and are also known and commercially available. They correspond to the formula R⁶CO—(OC₂H₄)ₘ—OR⁷, in which R⁶ is an alkyl and/or alkenyl group containing 7 to 21 carbon atoms, m is a number of 11 to 100 and R⁷ is a hydrogen atom or a CO—R⁶ group. The formula encompasses symmetrical (R⁶═R⁷) and asymmetrical compounds (R⁶; R⁷). Compounds of type (b)(iv) based on polyethylene glycols with molecular weights of 1,000 to 10,000, preferably 1,500 to 6,000 and more particularly 1500 to 3,000 are preferably used in the preparations according to the invention. Diester compounds of type (b)(iv) are particularly preferred. Besides compounds of type (b)(iv), polyglycols may also be present as secondary products from the production process.

Surface-Active Compounds of Type (b)(v)

These compounds are also known as alkyl (oligo)glycosides. Alkyl and alkenyl oligoglycosides are known nonionic surfactants which correspond to the formula R⁸O-[G]ₚ, in which R⁸ is an alkyl and/or alkenyl group containing 4 to 22 carbon atoms, G is a sugar unit containing 5 or 6 carbon atoms and p is a number of 1 to 10. They may be obtained by the relevant methods of preparative organic chemistry. The alkyl and/or alkenyl oligoglycosides may be derived from aldoses or ketoses containing 5 or 6 carbon atoms, preferably glucose. Accordingly, the preferred alkyl and/or alkenyl oligoglycosides are alkyl and/or alkenyl oligoglucosides. The index p in the general formula indicates the degree of oligomerization (DP), i.e., the distribution of mono- and oligoglycosides, and is a number of 1 to 10. Whereas p in a given compound must always be an integer and, above all, may assume a value of 1 to 6, the value p for a certain alkyl oligoglycoside is an analytically determined calculated quantity which is generally a broken number. Alkyl and/or alkenyl oligoglycosides having an average degree of oligomerization p of 1.1 to 3.0 are preferably used. Alkyl and/or alkenyl oligoglycosides having a degree of oligomerization of less than 1.7 and, more particularly, between 1.2 and 1.4 are preferred from the applicational point of view. The alkyl or alkenyl radical R⁸ may be derived from primary alcohols containing 4 to 11 and preferably 8 to 10 carbon atoms. Typical examples are butanol, caproic alcohol, caprylic alcohol, capric alcohol and undecyl alcohol and the technical mixtures thereof obtained, for example, in the hydrogenation of technical fatty acid methyl esters or in the hydrogenation of aldehydes from Roelen's oxosynthesis. Alkyl oligoglucosides having a chain length of C₈ to C₁₀ (DP=1 to 3), which are obtained as first runnings in the separation of technical C₈₋₁₈ coconut oil fatty alcohol by distillation and which may contain less than 6% by weight of C₁₋₂ alcohol as an impurity, and also alkyl oligoglucosides based on technical C_{9/11} oxoalcohols (DP=1 to 3) are preferred. In addition, the alkyl or alkenyl radical R⁸ may also be derived from primary alcohols containing 12 to 22 and preferably 12 to 14 carbon atoms. Typical examples are lauryl alcohol, myristyl alcohol, cetyl alcohol, palmitoleyl alcohol, stearyl alcohol, isostearl alcohol, oleyl alcohol, elaidyl alcohol, petroselinyl alcohol, arachyl alcohol, gadoleyl alcohol, behenyl alcohol, erucyl alcohol, brassidyl alcohol and technical mixtures thereof which may be obtained as described above. Alkyl oligoglucosides based on hydrogenated C_{12/14} cocoalcohol with a DP of 1 to 3 are preferred.

Surface-Active Compounds of Type (b)(vi)

Betaines are known surfactants which are mainly produced by carboxyalkylation, preferably carboxymethylation, of aminic compounds. The starting materials are preferably condensed with halocarboxylic acids or salts thereof, more particularly with sodium chloroacetate, one mol salt being formed per mol betaine. The addition of unsaturated carboxylic acids, such as acrylic acid for example, is also possible. Examples of suitable betaines are the carboxyalkylation products of secondary and, in particular, tertiary amines corresponding to formula (1):

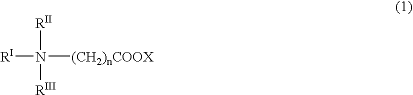

in which R^{I} stands for alkyl and/or alkenyl groups containing 6 to 22 carbon atoms, R^{II} stands for hydrogen or alkyl groups containing 1 to 4 carbon atoms, R^{III} stands for alkyl groups containing 1 to 4 carbon atoms, n is a number of 1 to 6 and X is an alkali metal and/or alkaline earth metal or ammonium. Typical examples are the carboxymethylation products of hexyl methyl amine, hexyl dimethyl amine, octyl dimethyl amine, decyl dimethyl amine, dodecyl methyl amine, dodecyl dimethyl amine, dodecyl ethyl methyl amine, C_{12/14} cocoalkyl dimethyl amine, myristyl dimethyl amine, cetyl dimethyl amine, stearyl dimethyl amine, stearyl ethyl methyl amine, oleyl dimethyl amine, C_{16/18} tallow alkyl dimethyl amine and technical mixtures thereof.

Other suitable betaines are carboxyalkylation products of amido-amines corresponding to formula (2):

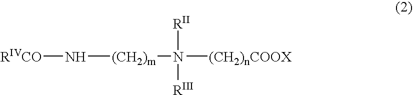

in which R^{IV}CO is an aliphatic acyl group containing 6 to 22 carbon atoms and 0 or 1 to 3 double bonds, m is a number of 1 to 3 and R^{II}, R^{III}, n and X are as defined above. Typical examples are reaction products of fatty acids containing 6 to 22 carbon atoms, namely caproic acid, caprylic acid, capric acid, lauric acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and technical mixtures thereof, with N,N-dimethyl aminoethyl amine, N,N-dimethyl aminopropyl amine, N,N-diethyl aminoethyl amine and N,N-diethyl aminopropyl amine which are condensed with sodium chloroacetate. It is preferred to use a condensation product of C_{8/18} cocofatty acid-N,N-dimethyl aminopropyl amide with sodium chloroacetate.

Other suitable starting materials for the betaines to be used in accordance with the invention are imidazolines corresponding to formula (3):

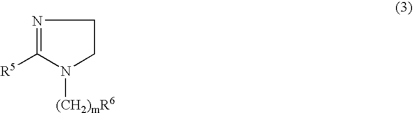

in which R^{V} is an alkyl group containing 5 to 21 carbon atoms, R⁶ is a hydroxyl group, an OCOR^{V} or NHCOR^{V} group and m=2 or 3. Imidazolines are also known compounds which may be obtained, for example, by cyclizing condensation of 1 or 2 mol of fatty acid with polyfunctional amines, for example aminoethyl ethanolamine (AEEA) or diethylene triamine. The corresponding carboxyalkylation products are mixtures of different open-chain betaines. Typical examples are condensation products of the above-mentioned fatty acids with AEEA, preferably imidazolines based on lauric acid or—again—C_{12/14} cocofatty acid which are subsequently betainized with sodium chloroacetate.
Surface-Active Compounds of Type (b)(vii)

These also known nonionic compounds are prepared, for example, by reacting alkyl epoxides with ethylene glycol and then with more ethylene oxide. They are also commercially available and correspond to general formula (IV):

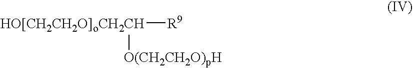

in which R⁹ is a linear or branched alkyl and/or alkenyl group containing 4 to 22 carbon atoms, o is a number of 1 to 20 and the index p is 0 or a number of 1 to 20.
Surface-Active Compounds of Type (b)(viii)

The compounds, which may also be termed hydroxy mixed ethers, correspond to general formula (V):

R¹⁰CH(OR¹¹)CH₂—OR¹¹ (V)

in which R¹⁰ is a saturated or unsaturated, branched or unbranched alkyl or alkenyl group containing 8 to 16 carbon atoms and the substituents R¹¹ independently of one another symbolize a group (CH₂CH₂O)ᵣCH₂CH(OH)R¹², in which r in each of the R¹¹ substituents independently stands for 0 or a number of 1 to 50 and R¹² is a saturated or unsaturated, branched or unbranched alkyl or alkenyl group containing 8 to 16 carbon atoms. These compounds are prepared, for example, by reacting compounds of formula (III) with more alkylene oxide having C chains of 8 to 18 carbon atoms under the conditions of an alkaline catalysis.
Surface-Active Compounds of Type (b)(ix)

These are nitrogen-containing compounds corresponding to general formula (VI):

NR¹³₃ (VI)

in which the substituents R¹³ independently of one another represent a group (CH₂CH₂O)ₛ—CH₂CH(OH)R¹⁴ or an alkyl group containing 8 to 16 carbon atoms and s in each substituent R¹³ independently represents 0 or a number of 1 to 50. Compounds of type (b)(ix) are obtainable, for example, by ethoxylation of alkylamines or triethanolamine and subsequent reaction with C₈₋₁₈ alkylene oxides under the conditions of alkaline catalysis.

Compounds (b)(i) to (b)(ix) may be individually combined with at least one compound of type a). Binary mixtures of a) and a compound of type b), more particularly a compound of type (b)(i) or (ii), are particularly preferred. Mixtures of various compounds of type b) may also be combined with HMEs of type a). In the case of mixtures containing several different compounds of type b), it can be of advantage to use these compounds in a ratio by weight of 1:1.

The compounds of type a) and b) are present alongside one another in a ratio by weight of 10:1 to 1:10 in the mixtures according to the invention. However, preferred mixtures can be those where the surface-active compounds of type a) and b) are present alongside one another in a ratio by weight of 5:1 to 1:5, preferably 3:1 to 1:3, more preferably 2:1 to 1:2 and most preferably 1:1. As already mentioned, the compounds of type (b)(i) to (b)(ix) may also be present alongside one another in any mixtures. However, the mixtures preferably consist of only one compound of type a) and one compound of type b).

The above-described mixtures are eminently suitable for use in cleaning preparations, more particularly in dish detergents and especially in automatic dish detergents. The mixtures may be used for improving the drying behavior and/or the clear rinse performance of cleaning preparations, more particularly dish detergents and especially automatic dish detergents. In principle, the solid preparations according to the invention may be produced by any of the methods known to the expert.

Such wax-like and preferably water-free or substantially water-free raw materials for use in cleaning preparations as described above, which—like the alkoxylates according to the invention—are obtained in the form of a melt in the synthesis process, can be converted into powders or granules, for example by the following processes known to the expert:
by spray cooling (prilling) of the melt in a spray tower
by pelleting of the melt on a cooling belt
by solidification of the melt in a fluidized bed
by conversion of the melt into droplets (for example using a Rieter “Droppo Line”; Brace microspheres; Gouda Jet Priller)
by solidification of the melt on cooling rollers.

In order to establish a desired particle size distribution, this process may optionally be followed by a grinding step.

The present invention also relates to cleaning preparations for hard surfaces containing surfactants, water softeners and optionally other ingredients, characterized in that the cleaning preparations contain alkoxylated fatty alcohols corresponding to general formula (I):

R—O—(CₙH₂ₙO)ₘ—H (I)

in which R is a linear, branched, saturated or unsaturated alkyl or alkenyl group containing at least 19 carbon atoms, m has a value of 20 or more and n stands for 2 or 3. The cleaning preparations according to the invention are ready-to-use cleaning preparations, preferably automatic dish detergents. They contain the alkoxylates of general formula (I) according to the invention preferably in quantities of 0.1 to 25% by weight, based on the total weight of the cleaning preparation. The cleaning preparations according to the invention additionally contain other surfactants preferably selected from the classes of polyethylene glycols, alkyl (oligo)glycosides, fatty alcohol alkoxylates except the compounds of formula (I), polyol hydroxyalkyl ethers and hydroxy mixed ethers. Relevant details can be found in the above description of compounds (b)(i) to (b)(ix) which are all suitable for use as surfactants either individually or in combination in the preparations according to the invention. The cleaning preparations preferably contain the mixtures of group a) and group b) surfactants described in the foregoing. Preferred preparations contain these surfactant mixtures in quantities of 0.1 to 50% by weight, preferably in quantities of 0.5 to 30% by weight and more particularly in quantities of 2 to 25% by weight.

The cleaning preparations themselves may additionally contain preferably phosphates and/or polyacrylates as water softeners, preferably in quantities of 1 to 80% by weight, based on the total weight of the cleaning preparation. The cleaning preparations may additionally contain enzymes, enzyme activators, bleaching agents, bleach boosters, complexing agents, disintegrators, dyes, biocides, solubilizers, perfumes or fragrances, inorganic salts, carriers, soil repellents, corrosion inhibitors and/or defoamers. Suitable inorganic or organic carriers are, for example, zeolites, alkali metal phosphates, alkali metal carbonates, alkali metal sulfates, alkali metal hydrogen carbonates, alkali metal silicates, alkali metal citrates, polysaccharides and derivatives thereof or polymers and mixtures thereof.

Preferred cleaning preparations according to the invention are present in solid form, i.e., as granules, flakes, powders, pellets or shaped bodies, such as tablets, more particularly as granules and shaped bodies. The solid cleaning preparations may be produced by such processes as grinding, flaking, pelleting, extrusion, spray crystallization, prilling and/or drying and granulation in thin layers (in a Flash Dryer). Several of these processes may optionally be combined with one another. A preferred embodiment uses the ethoxylates of general formula (I) as granules which are then formulated together with the other ingredients to produce the final cleaning preparation. Particularly preferred production processes for ready-to-use cleaning preparations are fluidized bed granulation and extrusion.

According to the invention, the cleaning preparations are preferably formulated as multifunctional preparations, i.e., they encompass cleaning agents, rinse agents and, optionally, water softening agents and/or metal cleaning or metal protecting agents in a solid supply form (so-called 2-in-1 or 3-in-1 or even 5-in-1 products).

Finally, the present invention relates to a liquid rinse agent containing at least water, a solubilizer and optionally an acid, preferably an organic acid, characterized in that compounds corresponding to general formula (I) are present in the rinse agent in quantities of 0.1 to 25% by weight. Suitable solubilizers are any of the corresponding compounds known to the expert, including for example short-chain alcohols, such as ethanol or propanol, or fatty acid esters and similar compounds. However, ethanol is particularly preferred.

Preferred organic acids are hydroxycarboxylic acids and especially citric acid or derivatives thereof, for example esters or alkoxylates of citric acid. Preferred liquid preparations contain the compounds of formula (I) in quantities of 0.2 to 20% by weight, preferably in quantities of 1.0 to 15% by weight and more particularly in quantities of 1.5 to 10% by weight. The liquid preparations may vary in their viscosity and, on the one hand, may be low in viscosity (such as water) or may be present in thickened form, for example in the form of oils or gels or in paste form.

### EXAMPLES

### 1. Clear Rinse Performance

Clear rinse performance was evaluated digitally using a process developed by Cognis for quantitatively measuring deposits on hard surfaces. In this process, which is described in European patent application 1 635 167 A1, smooth materials of glass, stainless steel, china and various plastics are washed in a domestic dishwasher under defined conditions (water with a hardness of 2°, 16° and 21° dH (depending on the particular application) and 50 g of a standard soil*). The washed items are then measured for spotting and filming in the measuring apparatus. The results are expressed as “distinctly better/better/same/worse than standard”.

The test results are set out in Table 1 below, tests I to III being comparison tests (=standard; formulation containing C16/18 fatty alcohol+40 EO) and formulations IV and V representing the Examples according to the invention.

The basis in each case was the following formulation for an automatic dish detergent (=ADD) of the 3-in-1 type (quantities in % by weight active substance):

| | |
|---|---|
| 7% by weight | sodium disilicate (SKS 6, Clariant) |
| 27.5% by weight | sodium carbonate |
| 4% by weight | polycarboxylate, sodium salt (Acusol ® 587) |
| 2.5% by weight | TAED |
| 8% by weight | sodium percarbonate |
| 2% by weight | ethoxylated fatty alcohol |
| to 100% by weight | sodium tripolyphosphate |

* standard soil: based on 1,000 g: mixture of 25 g each of ketchup, mustard and gravy, 300 g margarine, 150 g drinking milk, 15 g potato starch, 9 g egg yolk, 3 g benzoic acid, rest: water

TABLE 1

| Composition | I | II | III | IV | V |
|---|---|---|---|---|---|
| C16/18 fatty alcohol + 25 EO | | 2 | | | |
| C16/18 fatty alcohol + 40 EO | 2 | | | | |
| C22 fatty alcohol + 10 EO | | | 2 | | |
| C22 fatty alcohol + 25 EO | | | | 2 | |
| C22 fatty alcohol + 25 EO granules | | | | | 2 |

Clear rinse performance on

| | | | | | | |
|---|---|---|---|---|---|---|
| ++: distinctly better | cutlery | 0 | − | + | ++ | ++ |
| +: better | china | 0 | − | 0 | + | + |
| 0: comparable | glass | 0 | − | + | ++ | ++ |
| −: worse than standard | plastic | 0 | 0 | − | + | + |

It is clear from Table 1 that 3-in-1 ADD formulations IV and V according to the invention are far better in their performance properties than the comparison formulation. This is reflected in particular in their clear rinse performance on glass and stainless steel.

In another test, various ethoxylated fatty alcohols were added in quantities of 2% by weight to a commercially obtainable 3-in-1 ADD formulation and the clear rinse results were determined as described above. Test I represents the comparison product, tests II and II correspond to the invention.

TABLE 2

| | | | |
|---|---|---|---|
| Composition | I | II | III |

| | | | |
|---|---|---|---|
| C16/18 fatty alcohol + 25 EO | | | |
| C16/18 fatty alcohol + 40 EO | 2 | | |
| C22 fatty alcohol + 10 EO | | | |
| C22 fatty alcohol + 25 EO | | 2 | |
| C22 fatty alcohol + 25 EO granules | | | 2 |

Clear rinse performance on

| | | | | |
|---|---|---|---|---|
| ++: distinctly better | cutlery | 0 | + | + |
| +: better | china | 0 | 0 | + |
| 0: comparable | glass | 0 | + | + |
| −: worse than standard | plastic | 0 | 0 | 0 |

It is clear from Table 2 that the 3-in-1 ADD formulations according to the invention are better than the comparison formulation. This is reflected in particular in their clear rinse performance on glass and stainless steel.

Another test involved a commercially available 5-in-1 formulation for an ADD. Again, 2% by weight of ethoxylated fatty alcohol was added and the clear rinse results were determined as described above. The results are set out in Table 3.

TABLE 3

| | | | |
|---|---|---|---|
| Composition in % AS | I | II | III |

| | | | |
|---|---|---|---|
| C16/18 fatty alcohol + 25 EO | | | |
| C16/18 fatty alcohol + 40 EO | 2 | | |
| C22 fatty alcohol + 10 EO | | | |
| C22 fatty alcohol + 25 EO | | 2 | |
| C22 fatty alcohol + 25 EO granules | | | 2 |

Clear rinse performance on

| | | | | |
|---|---|---|---|---|
| ++: distinctly better | cutlery | 0 | + | ++ |
| +: better | china | 0 | 0 | 0 |
| 0: comparable | glass | 0 | + | + |
| −: worse than standard | plastic | 0 | 0 | 0 |

It is clear from Table 3 that 5-in-1 ADD formulations II and III according to the invention are far better than the comparison formulation. This is reflected in particular in their clear rinse performance on glass and stainless steel.

In another test, a commercially available 4-in-1 formulation was tested as described above (addition of 2% by weight fatty alcohol ethoxylate). The results of the clear rinse tests are set out in Table 4.

TABLE 4

| | | | |
|---|---|---|---|
| Composition in % AS | I | II | III |

| | | | |
|---|---|---|---|
| C16/18 fatty alcohol + 25 EO | | | |
| C16/18 fatty alcohol + 40 EO | 2 | | |
| C22 fatty alcohol + 10 EO | | | |
| C22 fatty alcohol + 25 EO | | 2 | |
| C22 fatty alcohol + 25 EO granules | | | 2 |

Clear rinse performance on

| | | | | |
|---|---|---|---|---|
| ++: distinctly better | cutlery | 0 | ++ | ++ |
| +: better | china | 0 | ++ | ++ |
| 0: comparable | glass | 0 | ++ | ++ |
| −: worse than standard | plastic | 0 | + | + |

It is clear from Table 4 that 4-in-1 ADD formulations II and III are better than comparison formulation 1. This is reflected in their clear rinse performance on all surfaces. By comparison with the incorporated melt, the granular form of the C22 fatty alcohol+25 EO tends to show better clear rinse performance on stainless steel, glass and ceramic.

### 2. Foaming Behavior

Foaming behavior was evaluated by the free-fall circulation method. 500 ml distilled water were poured into a double-walled 2-liter measuring cylinder of the free-fall circulation apparatus. The liquid was kept at 20° C.±1° C. 0.2 ml of the substance or formulation to be tested containing 0.2 ml active substance was pipetted into the pump-circulated water. At the same time, the stop watch was started. The total volume formed (foam and liquid) was recorded with the associated temperature after 30″, 1′, 2′, 3′, 5′, 10′, 20′ and 30′. In addition, the target temperature of the Julabo F12 MB thermostat was adjusted to 32° C. after 1 minute, to 45° C. after 10 minutes and to 55° C. after 20 minutes.

The results were expressed as “distinctly better/better/same/worse than” standard. The test results are set out in Table 5, I representing a comparison test (=standard; formulation containing C16/18 fatty alcohol+20 EO) and II and III the Examples according to the invention.

TABLE 5
Foaming behavior by the free-fall circulation method

| | | | |
|---|---|---|---|
| Composition in % AS | I | II | III |

| | | | |
|---|---|---|---|
| C16/18 fatty alcohol + 25 EO | 0.2 | | |
| C22 fatty alcohol + 25 EO | | 0.2 | |
| C22 fatty alcohol + 25 EO granules | | | 0.2 |

Foaming behavior after

| | | | | | |
|---|---|---|---|---|---|
| ++: distinctly weaker | 0.5 | min. | 0 | + | ++ |
| +: weaker | 2 | mins. | 0 | + | ++ |
| 0: comparable | 5 | mins. | 0 | + | ++ |
| −: stronger than standard | 10 | mins. | 0 | ++ | ++ |
| | 20 | mins. | 0 | ++ | ++ |

It is clear from Table 5 that the C22 fatty alcohol ethoxylates according to the invention foam far more weakly than the comparison product which is an important property for use in automatic dish detergents. This is apparent in particular after a time of more than 10 mins. at relatively high temperatures of 45 to 55° C. which corresponds to the conditions in the dishwasher. By comparison with the predissolved melt, the granular form of the C22 fatty alcohol+25 EO shows even weaker foaming behavior.

What is claimed is:

1. A cleaning preparation for cleaning hard surfaces, comprising:
at least one alkoxylated fatty alcohol corresponding to general formula (I):
R—O—(CₙH₂ₙO)ₘ—H (I)
in which R is a linear or branched, saturated or unsaturated, alkyl or alkenyl group containing at least 19 carbon atoms, m represents a value of 20 or more, and n represents 2 or 3.

2. The cleaning preparation according to claim 1, wherein R in general formula (I) is a linear or branched, saturated or unsaturated, alkyl or alkenyl group containing 19 to 30 carbon atoms.

3. The cleaning preparation according to claim 1, wherein m in general formula (I) represents a number from 20 to 30.

4. The cleaning preparation according to claim 1, wherein n in general formula (I) has a value of 2.

5. The cleaning preparation according to claim 1, wherein R is a linear, saturated alkyl group containing 22 carbon atoms, n represents a value of 2, and m represents a number from 22 to 26.

6. The cleaning preparation according to claim 1, wherein the compounds of general formula (I) are present in quantities of from about 0.1 to about 25% by weight, based on the weight of the cleaning preparation.

7. The cleaning preparation according to claim 1, further comprising a non-ionic surfactant selected from the group consisting of: polyethylene glycols, alkyl (oligo) glycosides, fatty alcohol alkoxylates which correspond to a formula different from general formula (I), polyol hydroxyalkyl ethers, hydroxy mixed ethers, and mixtures thereof.

8. The cleaning preparation according to claim 1, incorporated into a hard surface cleaner.

9. The cleaning preparation according to claim 1, incorporated into an automatic dish detergent or rinse agent.

10. A surfactant mixture, comprising:
(a) at least one compound corresponding to general formula (I):
R—O—(CₙH₂ₙO)ₘ—H (I)
in which R is a linear or branched, saturated or unsaturated, alkyl or alkenyl group containing at least 19 carbon atoms, m represents a value of 20 or more and n represents 2 or 3, and,
(b) at least one compound selected from the group consisting of:
(i) compounds corresponding to formula (IIa):
R¹O[CH₂CH₂O]ₓCH₂CH(OM)R² (IIa)
in which R¹ is a linear or branched, alkyl or alkenyl group containing 4 to 22 carbon atoms, or an R²—CH(OH)CH₂ group, in which R² is a linear or branched, alkyl or alkenyl group containing 8 to 16 carbon atoms, x represents a number from 40 to 80, and M is a hydrogen atom or a saturated alkyl group containing 1 to 18 carbon atoms;
(ii) compounds corresponding to formula (IIb):
R³O[CH₂CHCH₃O]_{y}[CH₂CH₂O]_{z}CH₂CH(OH)R⁴ (IIb)
in which R³ is a linear or branched, alkyl or alkenyl group containing 8 to 22 carbon atoms, R⁴ is a linear or branched, alkyl or alkenyl group containing 8 to 16 carbon atoms, y represents a number from 10 to 35, z is 0 or represents a value from 1 to 5, with the proviso that where R³═R¹, and at the same time R⁴═R², z must be at least 1;
(iii) ethoxylated fatty alcohols corresponding to general formula (III):
R⁵—(OC₂H₄)ₙ—OH (III)
in which R⁵ represents linear or branched, alkyl or alkenyl groups containing 8 to 22 carbon atoms, and z represents a number from 1 to 20;
(iv) compounds corresponding to general formula R⁶CO—(OC₂H₄)ₘ—OR⁷, in which R⁶ is an alkyl or alkenyl group containing 7 to 21 carbon atoms, m represents a number from 11 to 100 and R⁷ is a hydrogen atom or a CO—R⁶ group;
(v) alkyl (oligo)glycosides corresponding to the general formula R⁸⁰—[G]ₚ, in which R⁸ is an alkyl or alkenyl group containing 4 to 22 carbon atoms, G is a sugar unit containing 5 or 6 carbon atoms and p represents a number from 1 to 10;
(vi) betaines;
(vii) compounds corresponding to general formula (IV): 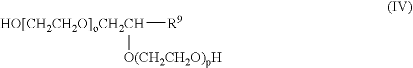
in which R⁹ is a linear or branched, alkyl or alkenyl group containing 4 to 22 carbon atoms, o represents a number from 1 to 20 and p is 0 or represents a number from 1 to 20;
(viii) compounds corresponding to general formula (V):
R¹⁰CH(OR¹¹)CH₂—OR¹¹ (V)
in which R¹⁰ is a saturated or unsaturated, branched or unbranched, alkyl or alkenyl group containing 8 to 16 carbon atoms and the substituents R¹¹ independently of one another represent a group (CH₂CH₂O)ᵣCH₂CH(OH)R¹², in which r in each of the R¹¹ substituents independently represents 0 or a number from 1 to 50 and R¹² is a saturated or unsaturated, branched or unbranched, alkyl or alkenyl group containing 8 to 16 carbon atoms; and
(ix) compounds corresponding to general formula (VI):
NR¹³₃ (VI)
in which the substituents R¹³ independently of one another represent a group (CH₂CH₂O)ₛ—CH₂CH(OH)R¹⁴, or an alkyl group containing 8 to 16 carbon atoms and s in each substituent R¹³ independently represents 0 or a number from 1 to 50.

11. The surfactant mixture according to claim 10, wherein the ratio by weight of components (a) and (b) ranges from about 10:1 to about 1:10, by weight of components (a) and (b).

12. The surfactant mixture according to claim 11, incorporated into a cleaning preparation for cleaning hard surfaces.

13. The surfactant mixture according to claim 12, wherein the mixture is present in quantities of from about 0.1 to about 50% by weight of the cleaning preparation.

14. The surfactant mixture according to claim 12, wherein component (a) is present in quantities of from about 0.1 to about 25% by weight of the cleaning preparation.

15. The surfactant mixture according to claim 12, wherein the cleaning preparation further comprises a water softener selected from the group consisting of: phosphates, polyacrylates, and mixtures thereof.

16. The surfactant mixture according to claim 15, wherein the water softener is present in quantities of from about 1 to about 80% by weight, by weight of the cleaning preparation.

17. The surfactant mixture according to claim 12, wherein the surfactant mixture is present in the cleaning preparation in the form of solid granules or solid shaped bodies.

18. The surfactant mixture according to claim 12, wherein the cleaning preparation is an automatic dish detergent.

19. A liquid rinse agent, comprising:
water;
a solubilizer; and
at least one alkoxylated fatty alcohol corresponding to general formula (I):
R—O—(CₙH₂ₙO)ₘ—H (I)
in which R is a linear or branched, saturated or unsaturated, alkyl or alkenyl group containing at least 19 carbon atoms, m represents a value of 20 or more, and n represents 2 or 3, wherein the at least one alkoxylated fatty alcohol corresponding to general formula (I) is present in quantities of from about 0.1 to about 25% by weight of the rinse agent.

20. The liquid rinse agent according to claim 19, further comprising an organic acid.
